(19) United States
(12) Patent Application Publication
(10) Pub. No.: US 2026/0263498 A1
(43) Pub. Date: Sep. 10, 2026

(54) USE OF FLUDARABINE IN TREATMENT OF METHICILLIN-RESISTANT STAPHYLOCOCCUS AUREUS INFECTIONS

(71) Applicant: Academy of Military Medical Sciences, Beijing (CN)

(72) Inventors: Bo MA, Beijing (CN); Zhi LI, Beijing (CN); Hua XU, Beijing (CN); Yulong LIU, Beijing (CN); Jianwei XIE, Beijing (CN)

(73) Assignee: Academy of Military Medical Sciences, Beijing (CN)

(21) Appl. No.: 19/642,678

(22) Filed: Apr. 9, 2026

Related U.S. Application Data
(63) Continuation of application No. PCT/CN2026/080245, filed on Feb. 27, 2026.

(30) Foreign Application Priority Data
Mar. 5, 2025 (CN) 202510251538.8

(51) Int. Cl. A61K 31/7076; A61P 31/04

(52) U.S. Cl. CPC A61K 31/7076; A61P 31/04

(57) ABSTRACT

A use of fludarabine in the treatment of methicillin-resistant Staphylococcus aureus (MRSA) infection is provided. In a mouse model of MRSA infection, it has been verified that fludarabine can improve the survival rate of MRSA-infected mice, reduce the bacterial load of MRSA in the lung, liver, spleen and kidney, and alleviate the degree of injury to the lung, liver, spleen and kidney caused by MRSA infection. It has also been found that PSMα3 is an important virulence factor of MRSA and is closely associated with the high pathogenicity of MRSA; PSMα3 can induce macrophage death in a concentration-dependent manner, and fludarabine can significantly reduce the cytotoxicity caused by PSMα3.

### CROSS REFERENCE TO THE RELATED APPLICATIONS

This application is a continuation application of International Application No. PCT/CN2026/080245, filed on Feb. 27, 2026, which is based upon and claims priority to Chinese Patent Application No. 202510251538.8, filed on Mar. 5, 2025, the entire contents of which are incorporated herein by reference.

### TECHNICAL FIELD

The present invention belongs to the technical field of biomedicine, and specifically relates to a use of fludarabine in the treatment of methicillin-resistant Staphylococcus aureus infections.

### BACKGROUND

The emergence and spread of the “superbug” methicillin-resistant Staphylococcus aureus (MRSA) worldwide, driven by the abuse of antibiotics, have posed immense difficulties and challenges for clinical anti-infective therapy and evolved into a major public health threat to human life and health. MRSA is the primary pathogenic bacterium responsible for healthcare-associated and community-acquired infections, and also a leading cause of severe suppurative infections, pneumonia, infective endocarditis, sepsis and septic shock. According to the US Centers for Disease Control and Prevention (CDC), approximately 20,000 people die from MRSA infections in the United States each year, and the mortality rate caused by MRSA infection ranks first among all clinically isolated drug-resistant bacteria. In China, the problem of MRSA infection is equally severe, with its clinical detection rate remaining above 30% for years, and it is also the main pathogenic bacterium leading to death from severe clinical infections. To date, the global number of deaths caused by MRSA infections has exceeded that of acquired immune deficiency syndrome (AIDS) and hepatitis B, making it the most serious of the world's three major infectious diseases [1,2]. Meanwhile, MRSA is multi-drug resistant and can resist the vast majority of clinically used antimicrobial agents, resulting in an extreme scarcity of clinical treatment strategies for MRSA. More critically, vancomycin, the “last line of defense” for the treatment of MRSA infections, has been overcome in recent years, and the emergence of vancomycin-resistant Staphylococcus aureus (VRSA) has pushed humanity into a dire predicament with no effective antibiotics available [3].

Phenol-soluble modulin α3 (PSMα3), an extremely important virulence factor of MRSA, can not only significantly enhance the pathogenicity of MRSA, but also help MRSA attack host immune cells to achieve immune evasion. Therefore, an in-depth investigation into the mechanism of action of PSMα3 during MRSA infection, and the clarification of its molecular mechanisms and key targets in regulating the body's immunity, are expected to become an important breakthrough for further revealing the pathogenic mechanism of MRSA and developing drugs to control MRSA infections [4].

With the deepening of research, researchers have found that the pathogenicity of bacteria is closely associated with the virulence factors they secrete. The strategy of attenuating bacterial virulence rather than directly killing bacteria can reduce bacterial pathogenicity and enhance the host's antibacterial immune response by selectively inhibiting the effector transmission of virulence factors, biofilm formation, immune evasion and other processes. Meanwhile, it exerts no selective survival pressure on bacteria and thus has the unique advantage of not inducing bacterial drug resistance. Therefore, identifying the key virulence factors of pathogenic bacteria, elucidating their unique mechanisms of action, and developing novel anti-virulence drugs have become a new trend in the research of new antibacterial drugs that do not induce drug resistance, as well as a new direction for the effective control of MRSA infections [5].

At present, the research and development costs of antibacterial drugs are rising steadily, and the development cycles are becoming increasingly prolonged. In the past decade, only a very small number of antibacterial drugs have been proven effective against MRSA-resistant strains in vitro, and their toxicity and pharmacokinetic characteristics remain unclear. More worryingly, the emergence of drug-resistant strains has far outpaced the development of new drugs. Moreover, once new antibacterial drugs are clinically applied, they will inevitably induce more severe bacterial drug resistance if they exert selective survival pressure on bacteria. Therefore, to effectively combat the increasingly severe MRSA-resistant bacterial infections, it is imperative to break through traditional approaches and explore new antibacterial strategies that do not induce drug resistance [6].

Fludarabine is an antineoplastic agent indicated for the treatment of patients with B-cell chronic lymphocytic leukemia (CLL) [7]. It is also used as a pre-transplant conditioning regimen. For instance, the study Large Pericardial Effusions as a Manifestation of Graft Versus Host Disease: a Single Institution Retrospective Study [8] reports a retrospective investigation into large pericardial effusion (LPE) as a manifestation of graft-versus-host disease (GVHD) following allogeneic hematopoietic stem cell transplantation, where the conditioning regimen included fludarabine in combination with busulfan or total body irradiation (TBI). Another study, Reduced Intensity Conditioning Matched Unrelated Donor Allograft for Kostmann Syndrome [9], presents a case report on matched unrelated donor (MUD) transplantation in a patient with Kostmann syndrome (congenital neutropenia), and the conditioning regimen mentioned in the document also incorporates fludarabine. To date, there have been no reports on the efficacy of fludarabine against methicillin-resistant Staphylococcus aureus infections.

Faced with the grim global situation of rampant MRSA infections, severe drug resistance, and a rising post-infection mortality rate, investigating the pathogenic mechanisms of MRSA and identifying novel drugs and strategies for the effective control of MRSA infections have become an urgent task that researchers must address.

### REFERENCES

1. Collaborators, G.B.D.A.R., Global burden of bacterial antimicrobial resistance 1990-2021: a systematic analysis with forecasts to 2050. Lancet, 2024. 404 (10459): p. 1199-1226.
2. van Hal, S. J., et al., Predictors of mortality in Staphylococcus aureus Bacteremia. Clin Microbiol Rev, 2012. 25 (2): p. 362-86.
3. McGuinness, W. A., N. Malachowa, and F. R. DeLeo, Vancomycin Resistance in Staphylococcus aureus Yale J Biol Med, 2017. 90 (2): p. 269-281.
4. Peschel, A. and M. Otto, Phenol-soluble modulins and staphylococcal infection. Nat Rev Microbiol, 2013. 11 (10): p. 667-73.
5. Rasko, D. A. and V. Sperandio, Anti-virulence strategies to combat bacteria-mediated disease. Nat Rev Drug Discov, 2010. 9 (2): p. 117-28.
6. Nazli, A., et al., Treatment of MRSA Infection: Where are We? Curr Med Chem, 2024. 31 (28): p. 4425-4460.
7. Ricci, F., et al., Fludarabine in the treatment of chronic lymphocytic leukemia: a review. Ther Clin Risk Manag, 2009. 5 (1): p. 187-207.
8. Norkin, M., et al., “Large Pericardial Effusions as a Manifestation of Graft Versus Host Disease: a Single Institution Retrospective Study”. Blood, 2009. 114 (22): p. 4659-4659.
9. Thachil, J., et al., Reduced Intensity Conditioning Matched Unrelated Donor Allograft for Kostmann Syndrome. Blood, 2005. 106 (11): p. 3854.

### SUMMARY

In the present application, it is discovered that fludarabine, when used for treating MRSA-infected mice, can improve the survival rate of the MRSA-infected mice, reduce the bacterial load of MRSA in the lung, liver, spleen and kidney, and alleviate the degree of injury to the lung, liver, spleen and kidney caused by MRSA infection. It is also found that PSMα3 is an important virulence factor of MRSA and is closely associated with the high pathogenicity of MRSA; PSMα3 can induce macrophage death in a concentration-dependent manner, and fludarabine can significantly reduce the cytotoxicity caused by PSMα3. The specific technical solutions of the present invention are as follows:

Aspect 1: Study on the Treatment of MRSA-Infected Mice with Fludarabine

A mouse model of MRSA infection was established using methicillin-resistant Staphylococcus aureus USA300 (abbreviated as “MRSA USA300”); a mouse model of Δpsmα3 infection was established using MRSA USA300 with the Δpsmα3 gene knocked out (abbreviated as “Δpsmα3”). Fludarabine was administered to mice via intraperitoneal injection at 1 h and 6 h after MRSA USA300 infection.

Results: At 2 days post-infection, all mice in the MRSA USA300-infected control group died, all mice in the normal control group survived, the survival rate of mice in the Δpsmα3-infected control group was 50%, and the survival rates of mice in the 10 mg/kg fludarabine treatment group, 20 mg/kg fludarabine treatment group and 50 mg/kg fludarabine treatment group were 30%, 40% and 80%, respectively.

At 24 h post-infection, compared with the MRSA USA300-infected control group, the logarithmic colony concentrations in the whole blood, liver, lung, spleen and kidney of mice in the Δpsmα3-infected control group were significantly reduced, with all P-values less than 0.001.

At 24 h post-infection, compared with the MRSA USA300-infected control group, the logarithmic colony concentrations in the whole blood, liver, lung, spleen and kidney of mice in the 50 mg/kg fludarabine treatment group were significantly reduced, with all P-values less than 0.001.

At 24 h post-infection, HE staining results of lung, liver, spleen and kidney tissues indicated the degree of organ injury in mice was ranked as follows: MRSA USA300-infected control group>Δpsmα3-infected control group>50 mg/kg fludarabine treatment group>normal control group.

Conclusion: PSMα3 is an important virulence factor of MRSA and is closely associated with the high pathogenicity of MRSA. Fludarabine significantly improves the survival rate of MRSA-infected mice, reduces the bacterial load in organs, and alleviates the degree of organ injury in infected mice, thereby exerting an anti-infective effect.

### Aspect 2: Study on PSMα3 as a Virulence Factor of MRSA

### (1) Evaluation of the Cytotoxicity of MRSA Virulence Factor PSMα3

After differentiating human acute monocytic leukemia cells (THP-1 cells) into mature macrophages, gradient solutions of PSMα3 were added. PSMα3 was found to induce macrophage death in a concentration-dependent manner, with a half-maximal inhibitory concentration (IC50) of 2.496±0.0513 μM.

### (2) Study on the Mechanism Underlying the Cytotoxicity of MRSA Virulence Factor PSMα3

Results: PSMα3 could activate the classic receptor-interacting protein kinase 1 (RIP1)/receptor-interacting protein kinase 3 (RIP3)/mixed lineage kinase domain-like protein (MLKL) signaling pathway of necroptosis downstream in a concentration-dependent manner and significantly increase the phosphorylation levels of RIP1, RIP3 and MLKL. The above results confirmed that PSMα3 induces macrophage necroptosis by activating the RIP1/RIP3/MLKL signaling pathway.

### Aspect 3: Evaluation of Inhibitory Effect of Fludarabine on PSMα3

### (1) In Vitro Evaluation of Inhibitory Effect of Fludarabine on PSMα3

Human acute monocytic leukemia cells (THP-1 cells) were differentiated into mature macrophages, followed by the addition of fludarabine and subsequent addition of PSMα3.

Results: PSMα3 caused the death of approximately 50% of macrophages; pre-treatment of cells with fludarabine significantly inhibited PSMα3-induced macrophage death and increased the cell survival rate to approximately 72%. Fludarabine could significantly reduce PSMα3-induced macrophage death.

### (2) In Vivo Evaluation of Inhibitory Effect of Fludarabine on PSMα3

Fludarabine treatment significantly reduced the expression level of phosphorylated-MLKL (p-MLKL), a key marker molecule of necroptosis, in pulmonary macrophages of mice infected with MRSA USA300. This indicated that fludarabine can block MRSA-induced macrophage necroptosis in vivo, maintain macrophage function and prevent MRSA immune evasion.

Advantages of the present application compared with the prior art:

The present application is the first to discover that fludarabine for the treatment of MRSA-infected mice can significantly reduce the bacterial load in organs, alleviate the degree of organ injury in infected mice, and improve the survival rate of infected mice. PSMα3 is a virulence factor of MRSA, and fludarabine can significantly reduce the cytotoxicity caused by PSMα3 both in vitro and in vivo, thus providing a new possibility for the treatment of MRSA infection.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: 7-day survival curve;

FIG. 2: Statistical comparison of logarithmic colony concentrations in the whole blood, liver, lung, spleen and kidney of mice in the MRSA USA300-infected control group, Δpsmα3-infected control group and 50 mg/kg fludarabine treatment group at 24 h post-infection;

FIG. 3: HE staining of lung, liver, spleen and kidney tissues of mice in the normal control group, USA300-infected control group, 50 mg/kg fludarabine treatment group and Δpsmα3-infected control group at 24 h post-infection;

FIG. 4: Survival curve of THP-1 cells treated with PSMα3;

FIG. 5: Western-Blot analysis results of the RIP1/RIP3/MLKL necroptosis signaling pathway promoted by MRSA virulence factor PSMα3;

FIG. 6: Inhibition of PSMα3-induced macrophage death by fludarabine;

FIG. 7: Inhibition of necroptosis in pulmonary macrophages of MRSA-infected mice by fludarabine.

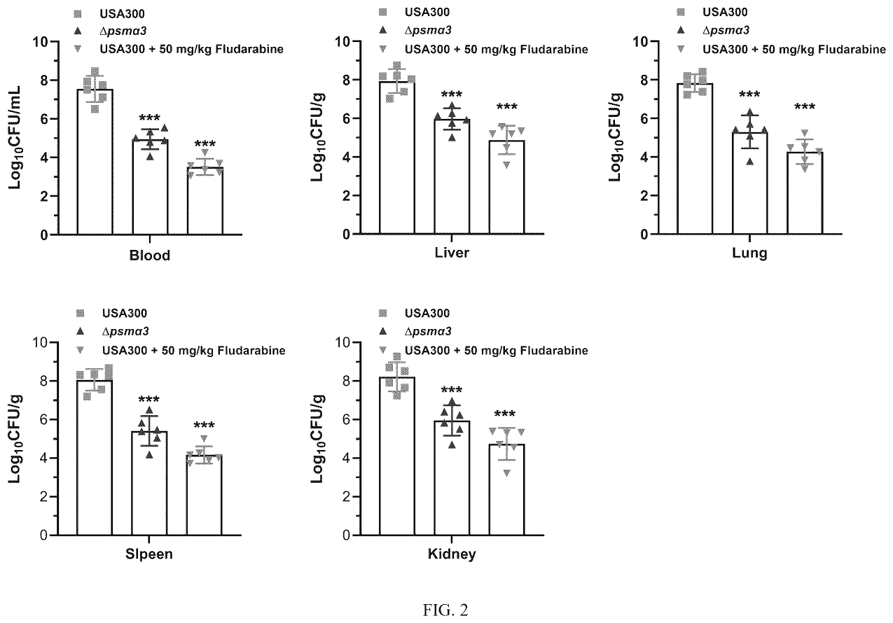

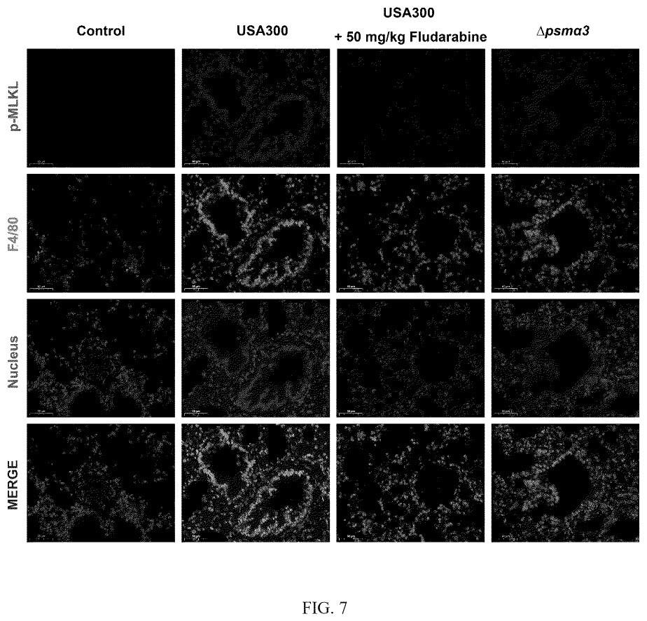

In FIG. 2 and FIG. 6,
*, ** and *** indicate a statistically significant difference between two groups, corresponding to P<0.05, P<0.01 and P<0.001, respectively.

### DETAILED DESCRIPTION OF THE EMBODIMENTS

The following examples are intended to illustrate the present invention, but not to limit its scope. Unless otherwise specified, the technical means used in the examples are conventional means well known to those skilled in the art.

The experimental methods used in the following examples are conventional methods unless otherwise stated.

All materials, reagents, etc., used in the following examples are commercially available unless otherwise specified.

Reagents and Materials

| Reagent or Material | Company | Cat. No. |
|---|---|---|
| MRSA standard strain USA300 | ATCC | — |
| PSMα3 (purity >98%) | Synpeptide Co., Ltd. | — |
| MRSA USA300 with Δpsma3 | Beijing Weijin | — |
| gene knockout | Biotech Co., Ltd. | |
| Human acute monocytic | ATCC | TIB-202 |
| leukemia cell line THP-1 | | |
| TSB medium | Solarbio | LA0110 |
| Sterile 1 × PBS | Gibco | 10010031 |
| phosphate-buffered solution | | |
| (pH = 7.4) | | |
| Sterile 10 × PBS | Gibco | 70011044 |
| phosphate-buffered solution | | |
| (pH = 7.4) | | |
| Fludarabine | Selleck | S1491 |
| DMSO | Sigma-Aldrich | D8418 |
| PEG300 | Merck | 807484 |
| Tween80 | Sigma-Aldrich | 63161 |
| Sterile deionized water | Biosharp | BL112A |
| BALB/c mice | SPF Biotechnology | |
| 1640 medium | Gibco | 10099141C |
| Fetal bovine serum (FBS) | Gibco | 10099141C |
| Penicillin/Streptomycin | Gibco | 15140122 |
| solution (100x) | | |
| β-Mercaptoethanol (55 mM) | Gibco | 21985023 |
| Phorbol 12-myristate | MCE | HY-18739 |
| 13-acetate (PMA) | | |
| CCK-8 reagent | Beyotime | C0043 |
| Phenylmethanesulfonyl | Beyotime | P2120 |
| fluoride (PMSF) solution | | |
| Phosphatase inhibitor cocktail | Beyotime | P1082 |
| SDS-PAGE precast gel | Beyotime | P0466S |
| (Tris-Gly, 4-15%, 15 wells) | | |
| 1 × TBST buffer | Solarbio | T1085 |
| PVDF membrane (0.45 μm) | Millipore | IPFL00010 |
| Rabbit anti-human | Cell Signaling | 44590 |
| p-RIP1 antibody | Technology | |
| Rabbit anti-human | Cell Signaling | 93654 |
| p-RIP3 antibody | Technology | |
| Rabbit anti-human | Cell Signaling | 91689S |
| | Technology | |
| p-MLKL antibody | | |
| Rabbit anti-human | Cell Signaling | 3493 |
| RIP1 antibody | Technology | |
| Rabbit anti-human | Cell Signaling | 13526 |
| RIP3 antibody | Technology | |
| Rabbit anti-human | Cell Signaling | 14993 |
| MLKL antibody | Technology | |
| GAPDH primary antibody | Cell Signaling | 2118S |
| | Technology | |
| Rabbit anti-mouse p-MLKL | Cell Signaling | 37333S |
| primary antibody | Technology | |
| Alexa Fluor 488-conjugated | Cell Signaling | 27076S |
| F4/80 fluorescent antibody | Technology | |
| HRP-conjugated goat | Abcam | ab205718 |
| anti-rabbit secondary antibody | | |
| Alexa Fluor 647-conjugated | Thermo | A21244 |
| goat anti-rabbit | | |
| secondary antibody | | |
| BCA Protein Assay Kit | Beyotime | P0012 |
| Chemiluminescent substrate kit | Abclonal | RM02867 |
| 4′,6-Diamidino-2-phenylindole | Beyotime | C1002 |
| (DAPI staining solution) | | |
| Ella Kit | Protein Simple | SPACKA-MP- |
| | | 003952 |

Instruments

| Instrument | Company | Model |
|---|---|---|
| Bacteria Incubator | Yiheng | DHP-9012 |
| CO₂ Incubator | SANYO | MCO-18AIC |
| UV-Vis Spectrophotometer | TECAN | Infinite M1000 pro |
| Centrifuge | Eppendorf | Centrifuge 5810R |
| Clean Bench | AIRTECH | BCM-1600A |
| Balance | Sartorius | SQP |
| Full-Wavelength | TECAN | M1000 pro |
| Multifunctional | | |
| Microplate Reader | | |
| Gel Imaging System | Protein Simple (USA) | FluorChem R |
| Biological Microscope | NIKON (Japan) | CI-S |
| Automated Microfluidic | Protein Simple (USA) | Ella |
| Immunoassay Analyzer | | |
| Microscope Slide Scanner | 3DHISTECH | Pannoramic |
| | | 250 FLASH |

### Example 1: In Vivo Therapeutic Effect of Fludarabine on MRSA Infection

### 1. Preparation of Bacterial Solution

Methicillin-resistant Staphylococcus aureus MRSA USA300 (hereinafter referred to as “MRSA USA300”) and MRSA USA300 with psmα3 gene knockout (hereinafter referred to as “Δpsmα3”) were inoculated into 10 mL of TSB medium, respectively, and cultured overnight at 37° C. on a shaker at 220 rpm. On the next day, the absorbance of the bacterial solution was measured at 630 nm. The concentration of the bacterial solution was adjusted to 1.5×10⁹ CFU/mL according to the conversion that an OD630 value of 0.1 corresponds to a bacterial solution concentration of approximately 1×10⁸ CFU/mL. The adjusted bacterial solution was centrifuged at 12,000 rpm at 4° C. for 10 min, and the supernatant was discarded. The bacteria were rinsed twice with sterile 1×PBS buffer, centrifuged at 12,000 rpm at 4° C. for 5 min each time, and the supernatant was discarded. Finally, the bacteria were resuspended in an equal volume of PBS buffer to maintain the concentration of the bacterial solution at approximately 1.5×10⁹ CFU/mL, thereby obtaining MRSA USA300 bacterial solution and Δpsmα3 bacterial solution.

### 2. Preparation of Fludarabine Solution

### (1) Fludarabine Working Solution

Approximately 80 mg of fludarabine powder was weighed using a balance. In a clean bench, the powder was dissolved in 1.6 mL of DMSO. After complete dissolution, 12.8 mL of PEG300 was added and mixed well, followed by 1.6 mL of Tween 80. After thorough mixing, 16 mL of sterile deionized water was added, and the mixture was inverted up and down to achieve uniformity, thus preparing a fludarabine working solution of 2.5 mg/mL.

### (2) Fludarabine Administration Solution

A 10 mL aliquot of fludarabine working solution was taken and diluted with 15 mL of DMSO-PEG300-Tween 80-sterile deionized water (5:40:5:50), followed by thorough mixing to obtain a fludarabine administration solution of 1 mg/mL.

3. Establishment of MRSA-Infected and Δpsmα3-Infected Mouse Models

Male BALB/c mice weighing 18-22 g were selected. A total of 96 mice were randomly divided into 6 groups with 16 mice per group, labeled as follows: normal control group, MRSA USA300-infected control group, 10 mg/kg fludarabine treatment group, 20 mg/kg fludarabine treatment group, 50 mg/kg fludarabine treatment group, and Δpsmα3-infected control group.

MRSA USA300-infected mouse model: Four groups of mice were injected intraperitoneally with 200 μL of MRSA USA300 bacterial solution (concentration approximately 1.5×10⁹ CFU/mL) to establish the MRSA USA300-infected mouse model.

Δpsmα3-infected mouse model: One group of mice was injected intraperitoneally with 200 μL of Δpsmα3 bacterial solution (concentration approximately 1.5×10⁹ CFU/mL) to establish the Δpsmα3-infected mouse model.

Normal control group: One group of mice received no bacterial solution injection and served as the normal control group.

### 4. Administration

The three fludarabine treatment groups were administered fludarabine administration solution twice, at 1 h and 6 h after MRSA USA300 infection. According to body weight, mice were intraperitoneally injected with 1.0 mg/mL fludarabine administration solution to achieve final fludarabine doses of 10 mg/kg (approximately 200 μL injected), 20 mg/kg (approximately 400 μL injected), and 50 mg/kg (approximately 1000 μL injected), respectively.

The recommended clinical dose of fludarabine phosphate for adults is 25 mg/m² body surface area per day for 5 consecutive days, with one intravenous cycle every 28 days. According to the body surface area conversion formula, the mouse dose is approximately 12.3 times the human dose. Therefore, the converted therapeutic dose of fludarabine in mice is approximately 307.5 mg/kg, and the median lethal dose (LD50) of fludarabine in mice is 375 mg/kg. The mouse administration doses of 10 mg/kg, 20 mg/kg, and 50 mg/kg used in this study were far lower than the converted therapeutic dose and median lethal dose of fludarabine in mice.

The MRSA USA300-infected control group, Δpsmα3-infected control group, and normal control group were injected with 200 μL of DMSO-PEG300-Tween 80-sterile deionized water (5:40:5:50).

### 5. Evaluation of In Vivo Therapeutic Effect of Fludarabine

### (1) Methods

The in vivo therapeutic effect of fludarabine was evaluated by organ CFU determination, tissue HE staining, and survival curve assay.

Survival curve assay: After administration, 10 BALB/c mice in each group were observed for 7-day survival, and survival curves were plotted and survival rates were calculated.

Organ CFU determination: At 24 h post-infection, 6 BALB/c mice from each group were anesthetized by intraperitoneal injection of sodium pentobarbital at a dose of 60 mg/kg. The left eyeball was removed, and blood was collected into anticoagulant tubes, a portion of which was placed on ice for later use. Mice were sacrificed by cervical dislocation, and parts of the liver, lung, spleen, and kidney were collected on ice, weighed, and homogenized. Subsequently, blood and tissue homogenates were serially diluted 10-fold in sterile TSB medium. A 100 μL aliquot of each dilution was evenly spread onto MHA agar plates. After overnight incubation at 37° C. in a constant-temperature incubator, the number of bacterial colonies (CFU) on each plate was counted, and the original colony count per gram of tissue (CFU/g) was calculated according to the dilution factor.

Tissue HE staining: Another portion of the liver, lung, spleen, and kidney was rinsed with sterile PBS buffer, then immersed in 10% paraformaldehyde fixative for 24 h. This was followed by gradient dehydration in 70%, 80%, 95%, and 100% ethanol for 30 min each, infiltration in xylene twice for 20 min each, and infiltration in paraffin twice for 12 min each. After paraffin embedding, 3 μm sections were prepared, deparaffinized, and subjected to HE staining. Tissue characteristics were observed using a biological microscope.

### (2) Results

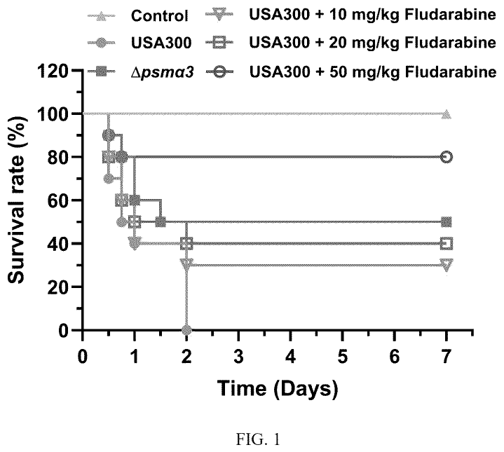

As shown in FIG. 1, 2 days after infection, all mice in the MRSA USA300-infected control group died (survival rate 0%), all mice in the normal control group survived (survival rate 100%), the survival rate of the Δpsmα3-infected control group was 50%, the survival rate of the 10 mg/kg fludarabine treatment group was 30%, the survival rate of the 20 mg/kg fludarabine treatment group was 40%, and the survival rate of the 50 mg/kg fludarabine treatment group was 80%.

As shown in FIG. 2, at 24 h post-infection, compared with the MRSA USA300-infected control group, the logarithmic colony concentrations in whole blood, liver, lung, spleen, and kidney were significantly decreased in the Δpsmα3-infected control group, with all P values less than 0.001.

At 24 h post-infection, compared with the MRSA USA300-infected control group, the logarithmic colony concentrations in whole blood, liver, lung, spleen, and kidney were significantly decreased in the 50 mg/kg fludarabine treatment group, with all P values less than 0.001.

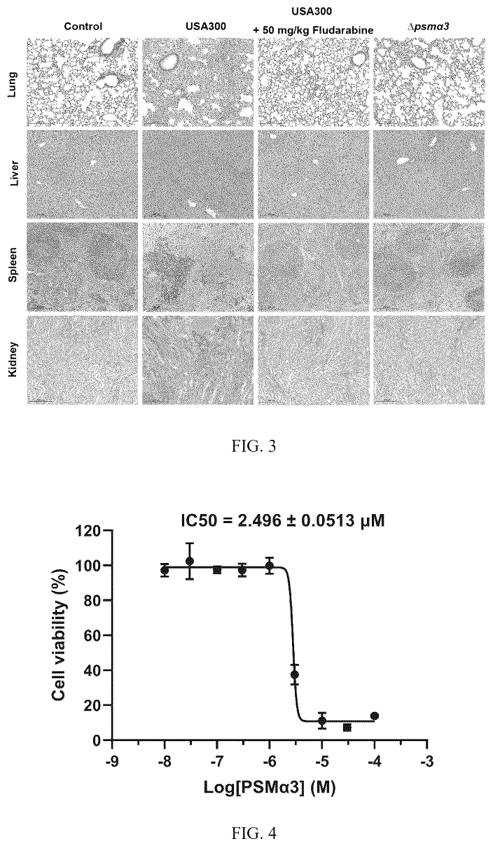

As shown in FIG. 3, HE staining of lung, liver, spleen, and kidney tissues at 24 h post-infection indicated that the degree of organ injury in mice was as follows: MRSA USA300-infected control group>Δpsmα3-infected control group>50 mg/kg fludarabine treatment group>normal control group.

### (3) Conclusion

PSMα3 is an important virulence factor of MRSA and is closely related to the high pathogenicity of MRSA. Fludarabine significantly improves the survival rate of MRSA-infected mice, reduces the bacterial load in organs, and alleviates the degree of organ injury in infected mice, thereby exerting an anti-infective effect.

### Example 2: Evaluation of the Cytotoxicity of MRSA Virulence Factor PSMα3

### 1. Culture of Human Acute Monocytic Leukemia Cells (THP-1 Cells)

THP-1 cells were cultured in RPMI 1640 medium supplemented with 10% fetal bovine serum (FBS), with the additional addition of 0.05 mM β-mercaptoethanol, 100 U/mL penicillin and 100 μg/mL streptomycin. The cells were cultured in a 5% CO₂ incubator at 37° C.

### 2. Evaluation of PSMα3 Cytotoxicity

### (1) Methods

Cultured THP-1 cells were prepared into a cell suspension with a concentration of 1×10⁵ cells/mL and seeded into a 96-well culture plate at 100 μL per well. Phorbol 12-myristate 13 -acetate was added to each well to a final concentration of 100 ng/ml, and the cells were induced for 24 h in a 5% CO₂ incubator at 37° C. to differentiate THP-1 cells into mature macrophages. After the induction, the culture supernatant was aspirated, and the cells were washed twice with serum-free RPMI 1640 medium. Then 100 μL of PSMα3 gradient solutions (diluted with serum-free RPMI 1640 medium to final concentrations of 0, 0.01, 0.03, 0.1, 0.3, 1, 3, 10, 30 and 100 μmol/L, with 3 replicate wells for each concentration) were added to the respective wells, and the cells were stimulated for 24 h in a 5% CO₂ incubator at 37° C. After the stimulation, the culture supernatant was aspirated, and 100 μL of RPMI 1640 medium containing 10% CCK-8 reagent was added to each well, followed by continuous incubation for 1 h at 37° C. The absorbance of each well was then measured at 450 nm using a full-wavelength multifunctional microplate reader.

$$Cell viability = [ ( experimental well - blank well ) / ( negative control well - blank well ) ] \times 100 \%$$

$$Negative control well : Untreated cells + RPMI 1640 medium + CCK - 8 reagent$$

$$Blank well : RPMI 1640 medium + CCK - 8 reagent$$

### (2) Results

As shown in FIG. 4, PSMα3 induced macrophage death in a concentration-dependent manner, with a half-maximal inhibitory concentration (IC50) of 2.496=0.0513 μM.

### Example 3: Mechanism Underlying the Cytotoxicity of MRSA Virulence Factor PSMα3

Cultured THP-1 cells were prepared into a cell suspension with a concentration of 1×10⁶ cells/mL and seeded into a 6-well culture plate at 1 mL per well. Phorbol 12-myristate 13-acetate was added to each well to a final concentration of 100 ng/mL, and the cells were induced for 24 h in a 5% CO₂ incubator at 37° C. to differentiate THP-1 cells into mature macrophages. After induction, the culture supernatant was aspirated, and the cells were washed twice with serum-free RPMI 1640 medium. Then 1 mL of PSMα3 gradient solutions (diluted with serum-free RPMI 1640 medium to final concentrations of 0, 1 and 3 μmol/L, with 3 replicate wells for each concentration) were added to the respective wells, and the cells were treated for 24 h in a 5% CO₂ incubator at 37° C. After the treatment, the culture supernatant was aspirated, and the cells were rinsed twice with 1×PBS buffer. Subsequently, 400 μL of cell lysis buffer (RIPA lysis buffer:PMSF:phosphatase inhibitor cocktail=100:1:1) was added to each well, and the cells were lysed by standing for 30 min in a 4° C. refrigerator. After complete lysis, the cell lysate was transferred to 1.5 mL EP tubes and centrifuged at 12,000 rpm for 20 min at 4° C.; the collected supernatant was the protein extract. The protein concentration of the sample from each group was determined using a BCA Protein Assay Kit.

Western blot assay was then performed to detect the expression and phosphorylation levels of RIP1, RIP3 and MLKL, the key molecules of the immune regulatory pathway in macrophages. 60 μL of protein sample from each group was mixed with 15 μL of 5×loading buffer, boiled in a water bath for 10 min, and set aside after cooling. SDS-PAGE precast gels (Tris-Gly, 4-15%, 15 wells) were used to assemble the electrophoresis system; protein samples and protein marker were loaded into the corresponding gel wells in sequence, with 25 μg of protein loaded per well for all groups. The power supply was turned on with the voltage initially set at 90 V; after the samples migrated out of the stacking gel, the voltage was increased to 120 V until the end of electrophoresis.

Subsequently, protein transfer was performed using a methanol-activated PVDF membrane and a transfer system at a constant voltage of 100 V for 100 min. The PVDF membrane was taken out and blocked with 5% non-fat milk powder at room temperature for 4 h, and incubated separately at 4° C. overnight with the primary antibodies of rabbit anti-human p-RIP1, rabbit anti-human p-RIP3, rabbit anti-human p-MLKL, rabbit anti-human RIP1, rabbit anti-human RIP3, rabbit anti-human MLKL and GAPDH (all diluted at a ratio of 1:1000 in 1×TBST). On the next day, the membrane was rinsed three times with 1×TBST for 10 min each time, then incubated with HRP-conjugated goat anti-rabbit secondary antibody and Alexa Fluor 647-conjugated goat anti-rabbit secondary antibody at room temperature for 1 h. After incubation, the membrane was rinsed three times with 1×TBST for 10 min each time. Finally, chemiluminescence development and imaging were performed using an Abclonal chemiluminescent substrate kit and a ProteinSimple gel imaging system.

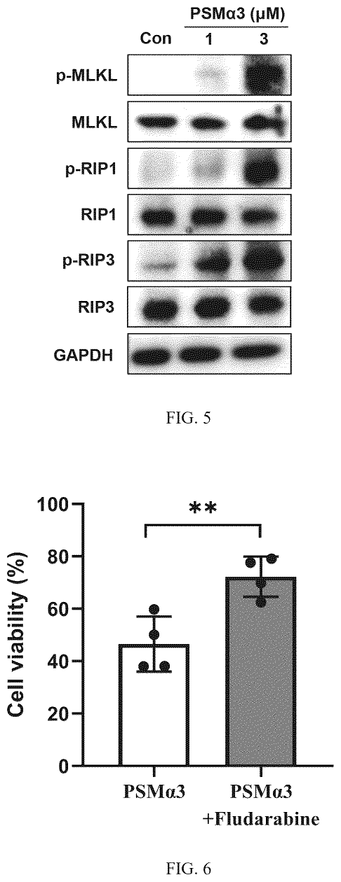

As shown in FIG. 5, PSMα3 activated the downstream classic RIP1/RIP3/MLKL necroptosis signaling pathway in a concentration-dependent manner and significantly upregulated the phosphorylation levels of RIP1, RIP3 and MLKL. These results confirmed that PSMα3 induces macrophage necroptosis by activating the RIP1/RIP3/MLKL signaling pathway.

### Example 4: Evaluation of the In Vitro Inhibitory Effect of Fludarabine on PSMα3

1. Culture of THP-1 Cells (same as Example 2)
2. In Vitro Assay for the Inhibitory Effect of Fludarabine on PSMα3-induced Cytotoxicity

Cultured THP-1 cells were prepared into a cell suspension with a concentration of 1×10⁵ cells/mL and seeded into a 96-well culture plate at 100 μL per well. Phorbol 12-myristate 13-acetate was added to each well to a final concentration of 100 ng/mL, and the cells were induced for 24 h in a 5% CO₂ incubator at 37° C. to differentiate THP-1 cells into mature macrophages. After induction, the culture supernatant was aspirated, and the cells were washed twice with serum-free RPMI 1640 medium. Then 100 μL of RPMI 1640 medium containing 0 or 5 μmol/L fludarabine was added to each well for 2 h of cell pretreatment. Subsequently, PSMα3 was added to a final concentration of 3 μmol/L, and the cells were stimulated for 24 h in a 5% CO₂ incubator at 37° C. After the stimulation, the culture supernatant was aspirated, and 100 μL of RPMI 1640 medium containing 10% CCK-8 reagent was added to each well, followed by continuous incubation for 1 h at 37° C. The absorbance of each well was then measured at 450 nm using a full-wavelength multifunctional microplate reader.

$$Cell viability = [ ( experimental well - blank well ) / ( negative control well - blank well ) ] \times 100 \%$$

$$Negative control well : Untreated cells + RPMI 1640 medium + CCK - 8 reagent$$

$$Blank well : RPMI 1640 medium + CCK - 8 reagent$$

As shown in FIG. 6, 3 μmol/L PSMα3 caused approximately 50% of macrophage death; pretreatment with fludarabine significantly inhibited PSMα3-induced macrophage death and increased the cell viability to approximately 72%. These results indicated that fludarabine can significantly reduce PSMα3-induced macrophage death.

### Example 5: Evaluation of the In Vivo Inhibitory Effect of Fludarabine on PSMα3

Paraffin sections of lung tissues from each group (Example 2) were deparaffinized and dehydrated with gradient ethanol, followed by antigen retrieval. The sections were then rinsed three times with phosphate-buffered saline with Tween 20 (1×PBST) for 5 min each time, and blocked with 10% goat serum in a humidified chamber at 37° C. for 30 min, followed by another three rinses with 1×PBST for 5 min each time. Alexa Fluor 488-conjugated F4/80 fluorescent antibody (diluted 1:50) for macrophage labeling was added dropwise to the tissue sections, which were then incubated overnight at 4° C. in a humidified chamber. The sections were rinsed three times with 1×PBST for 5 min each time, then incubated with rabbit anti-human p-MLKL primary antibody (diluted 1:500) at room temperature for 2 h, followed by another three rinses with 1×PBST for 5 min each time. Subsequently, Alexa Fluor 647-conjugated fluorescent secondary antibody was added dropwise and incubated at room temperature for 30 min. After rinsing with 1×PBST, the sections were stained with DAPI staining solution at room temperature for 30 min, rinsed again with 1×PBST, and mounted with anti-fade mounting medium. Imaging was performed using a microscope slide scanner.

As shown in FIG. 7, compared with the normal control group, the number of macrophages (labeled by F4/80) in the lung tissues of mice in the MRSA USA300 control group increased significantly, and the expression level of p-MLKL, a key marker molecule of necroptosis, was markedly upregulated; while in the Δpsmα3-infected control group, the expression level of p-MLKL was significantly lower than that in the MRSA USA300 control group. These results indicated that a large number of macrophages are recruited to the lesion site after MRSA USA300 infection, but MRSA USA300 can induce macrophage necroptosis through its virulence factor PSMα3, impair the bactericidal function of macrophages, and thus achieve immune evasion.

Treatment with fludarabine significantly reduced the expression level of p-MLKL, the key marker molecule of macrophage necroptosis, in the organs of MRSA USA300-infected mice. These findings demonstrated that fludarabine can block MRSA-induced macrophage necroptosis in vivo, maintain the normal function of macrophages, and prevent MRSA immune evasion.

Although the present invention has been described in detail with general explanations and specific embodiments above, modifications or improvements can be made thereto on the basis of the present invention, which will be obvious to those skilled in the art. Therefore, all such modifications and improvements made without departing from the spirit of the present invention fall within the scope of protection claimed by the present invention.

What is claimed is:

1. A use of fludarabine in preparation of a medicament against methicillin-resistant Staphylococcus aureus (MRSA), wherein the methicillin-resistant Staphylococcus aureus (MRSA) is MRSA USA300.

2. The use according to claim 1, wherein the fludarabine can improve a survival rate of mice infected with the MRSA USA300.

3. The use according to claim 1, wherein the fludarabine can reduce a bacterial load of the MRSA USA300 in a lung, liver, spleen, and kidney.

4. The use according to claim 1, wherein the fludarabine can alleviate a degree of injury to a lung, liver, spleen, and kidney caused by MRSA USA300 infection.

5. The use according to claim 1, wherein a key virulence factor of the MRSA USA300 is phenol-soluble modulin α3 (PSMα3).

6. The use according to claim 5, wherein the PSMα3 can induce macrophage death in a concentration-dependent manner.

7. The use according to claim 5, wherein the PSMα3 induces macrophage necroptosis by activating a receptor-interacting protein kinase 1 (RIP1)/receptor-interacting protein kinase 3 (RIP3)/mixed lineage kinase domain-like protein (MLKL) signaling pathway.

8. The use according to claim 5, wherein the fludarabine can reduce an expression level of phosphorylated-MLKL (p-MLKL), a key marker molecule of macrophage necroptosis, in lungs of mice infected with the MRSA USA300.
